(19) United States
(12) Patent Application Publication
(10) Pub. No.: US 2018/0266975 A1
(43) Pub. Date: Sep. 20, 2018

(54) BIOSENSOR USING DNA-BASED CONDUCTIVE NANOWIRE AND METHOD FOR MANUFACTURING THE SAME

(71) Applicant: Gumi Electronics & Information Technology Research Institute, Gyeongsangbuk-do (KR)

(72) Inventor: Hyung Jin Kim, Gyeongsangbuk-do (KR)

(21) Appl. No.: 15/474,384

(22) Filed: Mar. 30, 2017

(30) Foreign Application Priority Data
Mar. 20, 2017 (KR) 10-2017-0034791

(51) Int. Cl. G01N 27/04; G01N 33/543; C23C 16/50

(52) U.S. Cl. CPC G01N 27/041; C23C 16/50; G01N 33/5438

(57) ABSTRACT

A biosensor using a DNA-based conductive nanowire and a method for manufacturing the same are disclosed, and more particularly, a biosensor using a DNA-based conductive nanowire, which includes a DNA-based conductive nanowire formed by coating with conductive nanoparticles having spontaneous positive charges and a protein detection receptor coupled to the DNA-based conductive nanowire by electrostatic attraction to determine diseases at high sensitivity, and a method for manufacturing the same. A method for manufacturing a biosensor using a DNA-based conductive nanowire includes a DNA alignment process of selectively aligning DNA on a substrate, a DNA-based conductive nanowire manufacturing process in which conductive nanoparticles charged by positive charges are coupled to the aligned DNA to manufacture the DNA-based conductive nanowire having spontaneous positive charges, and a protein detection receptor fixing process of fixing a receptor for detecting proteins to the DNA-based conductive nanowire.

### TECHNOLOGICAL FIELD

The present disclosure relates to a biosensor using a DNA-based conductive nanowire and a method for manufacturing the same, and more particularly, to a biosensor using a DNA-based conductive nanowire, which includes a DNA-based conductive nanowire formed by coating with conductive nanoparticles having spontaneous positive charges and a protein detection receptor coupled to the DNA-based conductive nanowire by electrostatic attraction to determine diseases at high sensitivity, and a method for manufacturing the same.

### BACKGROUND

In general, a biosensor includes a bioreceptor selectively reacting with and coupled to a specific material and a signal transducer converting a signal generated by being coupled to the bioreceptor to confirm whether a specific bio-material such as protein, gene, hormone, and virus exists.

Since biosensors are usefully used in the fields of food safety research and environmental monitoring in addition to its application in the fields of medical diagnostics, needs for research and development of the biosensors is increasing. Thus, studies to overcome the limitations of the existing technologies and improve sensor characteristics such as rapidity, high sensitivity, and high selectivity have to be accompanied. The most notable area of the high-performance sensor technologies is sensitivity improvement.

Nano-scale materials are emerging as a very important area of research in recent years because of having new physical and chemical properties such as unique electrical, optical, and mechanical properties. Also, studies on nanostructures that have been carried out so far show possibility as new element materials in the future. Particularly, since nano-scale elements are small in size to increase in surface area/volume ration, the nano-scale elements may be applied to various kinds of sensors because electrical and chemical reaction occurring on a surface thereof becomes dominant.

As examples of biosensors to which nano technologies are grafted, a biosensor using a silicon nanowire, a method for manufacturing the biosensor using the silicon nanowire, and a method for detecting a specific cell by using the biosensor are disclosed in Korean Patent Registration No. 10-1085879. However, the silicon nanowire having electrical conductivity reacts only within a relatively narrow concentration range, and it is difficult to detect a target material having a low concentration, and thus, it is difficult to detect biomaterials at high sensitivity.

### SUMMARY

The presently described embodiments solve the abovementioned problems, and an object thereof is to provide a biosensor using a DNA-based conductive nanowire, which includes a DNA-based conductive nanowire formed by coating with conductive nanoparticles having spontaneous positive charges and a protein detection receptor coupled to the DNA-based conductive nanowire by electrostatic attraction to determine diseases at high sensitivity, and a method for manufacturing the same.

To achieve the above-described object, an embodiment provides a method for manufacturing a biosensor using a DNA-based conductive nanowire, the method including: a DNA alignment process (S100) of selectively aligning DNA on a substrate; a DNA-based conductive nanowire manufacturing process (S200) in which conductive nanoparticles charged by positive charges are coupled to the aligned DNA to manufacture the DNA-based conductive nanowire having spontaneous positive charges; and a protein detection receptor fixing process (S300) of fixing a receptor for detecting proteins to the DNA-based conductive nanowire.

The DNA alignment process (S100) may include: a photoresist layer formation process (S110) of forming a photoresist layer that controls a line width of a photoresist pattern to a thickness of about 1 nm to about 10 nm after the photoresist pattern is formed on the substrate; a nanomaterial adsorption inhibitor coating process (S120) of applying a nanomaterial adsorption inhibitor for preventing a nanomaterial from being adsorbed onto a photoresist pattern non-formation area of the substrate on which the photoresist layer is formed; a nanomaterial adsorbent coating process (S130) in which the photoresist pattern formed on the substrate is removed, and a nanomaterial adsorbent charged by the positive charges is applied to the area of the substrate from which the photoresist pattern is removed; and a DNA fixation process (S140) of fixing the DNA having negative charges to the substrate coated with the nanomaterial adsorbent.

In the photoresist layer formation process (S110), the line width may be controlled through a plasma downstream-type ashing process.

In the nanomaterial adsorption inhibitor coating process (S120), the nanomaterial adsorption inhibitor may include octadecyltrichlorosilane (OTS) or diamond like carbon (DLC).

In the nanomaterial adsorbent coating process (S130), the nanomaterial adsorbent may include aminopropyltriethoxysilane (APS) charged by the positive charges.

In the DNA fixation process (S140), the substrate coated with the nanomaterial adsorbent may be slanted to allow a solution containing the DNA to flow and thereby to fix the DNA.

In the DNA fixation process (S140), the substrate coated with the nanomaterial adsorbent may be immersed into a solution containing the DNA and then takes out to fix the DNA.

In the DNA-based conductive nanowire manufacturing process (S200), the aligned DNA and the conductive nanoparticles charged by the positive charges may be coupled to each other by electrostatic attraction.

The conductive nanoparticles charged by the positive charges may be spontaneously functionalized by an amine group.

The conductive nanoparticles charged by the positive charges may include at least one of metal particles, semiconductor particles, magnetic particles, polymer particles.

The method may further include an electrode formation process of connecting a source electrode coming into electrical contact with the DNA-based conductive nanowire to a drain electrode disposed to be spaced apart from the source electrode.

The protein detection receptor may include one of biotin, anti-AFP, and anti-PIVKA-II.

To achieve the above-described object, another embodiment provides a biosensor using the DNA-based conductive nanowire manufactured.

The accompanying drawings are included to provide a further understanding of the inventive concept, and are incorporated in and constitute a part of this specification. The drawings illustrate exemplary embodiments of the inventive concept and, together with the description, serve to explain principles of the inventive concept.

### BRIEF DESCRIPTION OF THE FIGURES

FIGS. 1(a) to 1(e) are conceptual views illustrating a DNA alignment order in a DNA alignment process (S100),

FIGS. 2(a) and 2(b) are views for comparing accuracy in coupling between a protein detection receptor (biotin) and a target material (streptavidin) when a photoresist pattern has a line width of about 100 nm and when the photoresist pattern has a line with of about 10 nm,

FIG. 3 is a conceptual view illustrating a process of forming a nanowire in a DNA-based conductive nanowire manufacturing process (S200),

FIG. 4 is a conceptual view illustrating a state in which the protein detection receptor is fixed to the DNA-based conductive nanowire in a protein detection receptor fixing process (S300), and

FIGS. 5(a) to 5(b) are views illustrating a variation in electrical characteristic due to whether the target material exists and a concentration of the target material according to an embodiment.

### DETAILED DESCRIPTION

Hereinafter, specific characteristics and advantages will be described in detail with reference to the accompanying drawings. Moreover, detailed descriptions related to well-known functions or configurations will be ruled out in order not to unnecessarily obscure the contemplated subject matter.

The presently described embodiments relate to a biosensor using a DNA-based conductive nanowire and a method for manufacturing the same, and more particularly, to a biosensor using a DAN-based conductive nanowire, which includes a DNA-based conductive nanowire formed by coating with conductive nano particles having spontaneous positive charges and a protein detection receptor coupled to the DNA-based conductive nanowire by electrostatic attraction to determine diseases at high sensitivity, and a method for manufacturing the same.

The method for manufacturing the biosensor using the DNA-based conductive nanowire includes a DNA alignment process (S100) of selectively aligning DNA on a substrate, a DNA-based conductive nanowire manufacturing process (S200) in which conductive nanoparticles charged by positive charges are coupled to the aligned DNA to manufacture the DNA-based conductive nanowire having spontaneous positive charges, and a protein detection receptor fixing process (S300) of fixing a receptor for detecting proteins to the DNA-based conductive nanowire.

The DNA alignment process (S100) may be a process of selectively aligning the DNA on the substrate and include a photoresist layer formation process (S110) of forming a photoresist layer that controls a line width of a photoresist pattern to a thickness of about 1 nm to about 10 nm after the photoresist pattern is formed on the substrate, a nanomaterial adsorption inhibitor coating process (S120) of applying a nanomaterial adsorption inhibitor for preventing a nanomaterial from being adsorbed onto a photoresist pattern non-formation area of the substrate on which the photoresist layer is formed, a nanomaterial adsorbent coating process (S130) in which the photoresist pattern formed on the substrate is removed, and a nanomaterial adsorbent charged by the positive charges is applied to the area of the substrate from which the photoresist pattern is removed, and a DNA fixation process (S140) of fixing the DNA having negative charges to the substrate coated with the nanomaterial adsorbent.

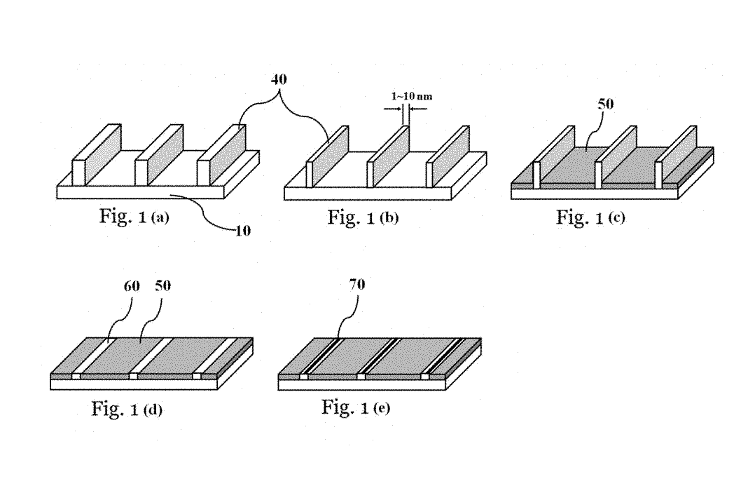

FIG. 1(a) to (e) are conceptual views illustrating a DNA alignment order in the DNA alignment process (S100).

The photoresist layer formation process (S110) may be a process of controlling the line width of the photoresist pattern in a nano unit after forming the photoresist pattern 40 on the substrate 10.

FIG. 1(a) illustrates a state in which the photoresist pattern is formed on the substrate. A line width in a micrometer unit may be adjusted to the line width in the nano unit by using a plasma downstream-type ashing process as illustrated in FIG. 1(b).

The process of forming the photoresist pattern 40 may be performed by using UV lithography, X-ray lithography, E-beam lithography, or ion lithography, more preferably, the E-beam lithography through which the pattern is capable of being more accurately formed.

Here, the pattern may not be formed in a simple linear shape, but be formed in a cross or lattice shape.

Here, the line width of the photoresist pattern 40 is adjusted in a nano unit of about 1 nm to about 10 nm. When the photoresist pattern 40 has a line width of about 1 nm or less, it may be difficult to secure a sufficient space for adsorbing the DNA. As a result, it may be difficult to fix the nanoparticles coupled to the DNA to the protein detection receptor coupled to the nanoparticles, and thus, it may be difficult to expect the confirmation of the target material to be detected and the detection of the target material at the high sensitivity.

The protein detection receptor and a target material to be detected (hereinafter, referred to as a detection target material) have to be coupled to a surface of the nanowire formed in the DNA-based conductive nanowire manufacturing process (S200) that will be described later. When the photoresist pattern 40 has a line width exceeding about 10 nm, since the formed nanomaterial adsorbent has a width that is relatively greater than a size of each of the protein detection receptor and the detection target material, the nanomaterial adsorbent may not be coupled to the surface of the nanowire, but be coupled to a portion coated with the nanomaterial adsorbent, on which the nanowire is not formed, thereby deteriorating accuracy.

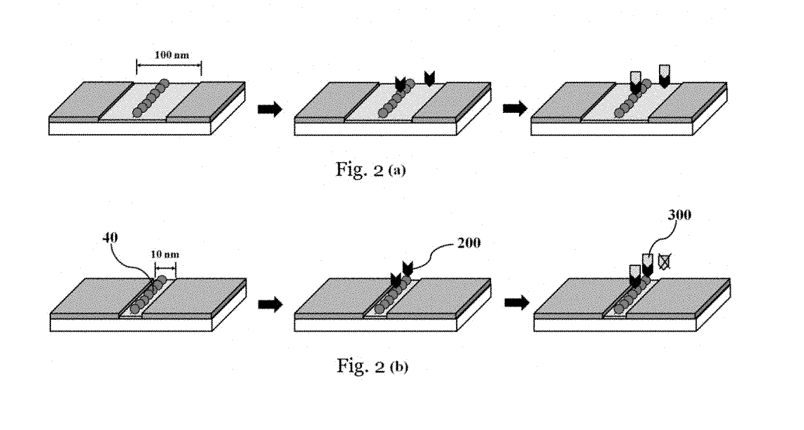

FIGS. 2(a) and 2(b) are views for comparing accuracy in coupling between the protein detection receptor (biotin) and the target material (streptavidin) according to the line width of the photoresist pattern. FIG. 2(a) is a view when the photoresist pattern has a line width of about 100 nm, and FIG. 2(b) is a view when the photoresist pattern has a line width of about 10 nm.

When the nanowire is formed by using the conductive nanoparticles having a mean particle diameter of about 5 nm, and the biotin and the streptavidin, which have the nano size (the mean particle diameter of about 5 nm), are coupled to the surface of the nanowire, if the photoresist pattern has a line width exceeding about 10 nm, the photoresist pattern may be formed with an area relatively greater than a size of each of the biotin and the streptavidin. Thus, the biotin and the streptavidin may be attached to a portion on which the nanowire is not formed to deteriorate the detection and sensitivity of the target material.

On the other hand, when the photoresist pattern 40 has a line width of about 10 nm or less, the unnecessary attachment of the biotin may be prevented to improve the detection and sensitivity of the target material.

The line width may be adjusted by using the plasma downstream-type ashing process. The ashing process using plasma may be a process of etching the photoresist pattern 40 by using a plasma generation device. Here, the more the exposure time of the photoresist pattern 40 to the plasma increases, the more the line width of the photoresist pattern 40 decreases. The ashing process using the plasma may be performed for about 3 minutes to about 6 minutes with respect to an initial line width of about 18 nm to about 25 nm to prevent the line width of the photoresist pattern 40 from being excessively narrowed or prevent the photoresist pattern 40 from being broken. The exposure time may vary according to the initial line width.

In the nanomaterial adsorption inhibitor coating process (S120), a nanomaterial adsorption inhibitor 50 is applied to the photoresist pattern non-formation area of the substrate on which the photoresist layer is formed (see FIG. 1(c)).

Here, the ‘nanomaterial’ represents the DNA and a material 70 containing the DNA.

The material 70 containing the DNA may be a solution and dispersion liquid, which contain the DNA.

The nanomaterial adsorption inhibitor 50 may be a composite that is applied to prevent the nanomaterial, i.e., the DNA and the material 70 containing the DNA from being adsorbed onto the area of the substrate on which the photoresist pattern 40 is not formed. Exemplary examples of the nanomaterial adsorption inhibitor 50 may include octadecyltrichlorosilane (OTS) or diamond like carbon (DLC), but is not limited thereto.

The OTS may be formed through a liquid coating method, and the DLC may be formed through an RF (radio frequency) plasma enhanced chemical vapor deposition (PECVD) method using methane and hydrogen gases.

In the nanomaterial adsorbent coating process (S130), the photoresist pattern 40 formed on the substrate may be removed to apply the nanomaterial adsorbent 60 charged by the positive charges to the area of the substrate from which the photoresist pattern 40 is removed (see FIG. 1(d)).

Aminopropyltriethoxysilane (APS) charged by the positive charges may be used as the nanomaterial adsorbent 60. Thus, the DNA having the negative charges and the nanomaterial 70 containing the DNA may be coupled to each other by electrostatic attraction.

The DNA fixation process (S140) may be a process of fixing the nanomaterial 70 containing the DNA having the negative charges to the substrate coated with the nanomaterial adsorbent. In this process, the nanomaterial 70 containing the DNA having the negative charges may be coupled to the APS, which is coated in the previous process and charged by the positive charged, through the electrostatic attraction (see FIG. 1(e)).

Here, in order to selectively locate the DNA and the nanomaterial 70 containing the DNA at a specific position of the substrate coated with the nanomaterial adsorbent 60 or align the DNA and the nanomaterial 70 in a specific direction, the substrate coated with the nanomaterial adsorbent may be slanted to allow the solution containing the DNA to flow and thereby to fix the DNA, or the substrate coated with the nanomaterial adsorbent may be immersed into the solution containing the DNA and then be pulled in a specific direction to allow the solution containing the DNA to flow in a predetermined direction and thereby fix the DNA.

Furthermore, when the nanomaterial is adsorbed onto the pattern that does not have the simple linear shape, but have the lattice shape, a process of slanting the substrate in a different direction that rotates at an angle of about 90° or a process of pulling the substrate in a different direction that rotates at an angle of about 90° may be additionally performed.

Thereafter, a cleaning process of cleaning the substrate may be performed to remove the DNA-containing solution and the nanomaterial, which are adsorbed onto the area of the substrate except for the area of the substrate to which the nanomaterial adsorbent is applied.

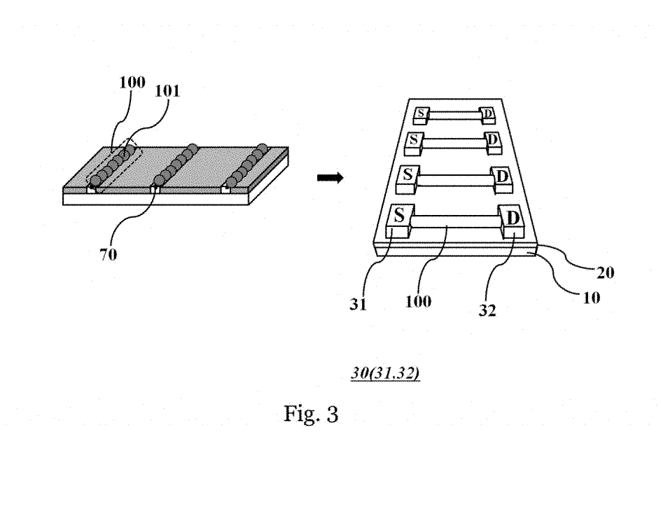

FIG. 3 is a conceptual view illustrating a process of forming the nanowire in the DNA-based conductive nanowire manufacturing process (S200).

The DNA-based conductive nanowire manufacturing process (S200) may be a process of coupling conductive nano particles 101, which are charged by positive charges, to the aligned DNA 70 to manufacture a DNA-based conductive nanowire 100 having spontaneous positive charges.

The conductive nanoparticles may be particles selected from the group consisting of metal particles, semiconductor particles, magnetic particles, polymer particles, or a combination thereof. For example, the conductive nanoparticles may be made of gold (Au), silver (Ag), platinum (Pt), aluminum (Al), copper (Cu), palladium (Pd), titanium (Ti), and the like, but is not limited thereto.

More particularly, when gold nanoparticles are used, since various organic molecules are strongly coupled to surfaces of the gold nanoparticles, the stable coupling state may be maintained at a physiological salt concentration at which biomaterials (oligonucleotide, proteins, and the like) are capable of being maintained in its original structure. Thus, more superior sensitivity and quick and easy analysis may be enabled to realize high reproducibility.

Each of the conductive nanoparticles may have a size of about 1 nm to about 50 nm. In the above-described range, the coupling with the protein detection receptor may be superior, and high sensitive sensing may be enabled.

The conductive nanoparticles may be charged by the positive charges. Thus, the conductive nanoparticles may be coupled to the DNA having the negative charges through the electrostatic attraction.

The conductive particles charged by the positive charges may be spontaneously functionalized by an amine group to improve affinity and the electrostatic attraction with the protein detection material through the induction of the amine group.

The introduction of the amine group to the surface may be performed through a liquid coating method, in which the nanoparticles are immersed into a solution containing the amine group or coated with the solution, or a nitrogen (N₂) plasma treating method, but is not limited thereto.

Although a compound containing the amine group is not limited, a silane coupling agent containing the amine group or a compound having a thiol group and the amine group may be used.

Exemplary examples of the silane coupling agent containing the amine group may be a compound including at least one of materials consisting of 3-aminopropyltrimethoxysilane, 3-aminopropyltrimethoxysilane, 3-(2-aminoethyl) aminopropyltrimethoxysilane, 3-anilinopropyltrimethoxysilane, N-phenyl-3-aminopropyltrimethoxysilane, and a combination thereof, but is not limited thereto.

Exemplary examples of the compound having the thiol group and the amine group may be one of compounds consisting of 2-aminoethane thiolhydrochloride, 4-aminothiolphenol, 6-amino-1-hexanethiolhydrochloride, and a combination thereof, but is not limited thereto.

The compound containing the amine group may be mixed with a solvent to react with nanoparticles, which are in a solution state. The nanoparticles may be immersed into the solution containing the amine group at a predetermined temperature (about 20° C. to about 150° C.) for a predetermined time (about 2 hours to about 48 hours) to react with the solution.

A self-assembled monolayer (SAM) may be spontaneously formed on the surfaces of the conductive nano particles through the above-described method to improve the affinity and the electrostatic attraction with the protein detection material.

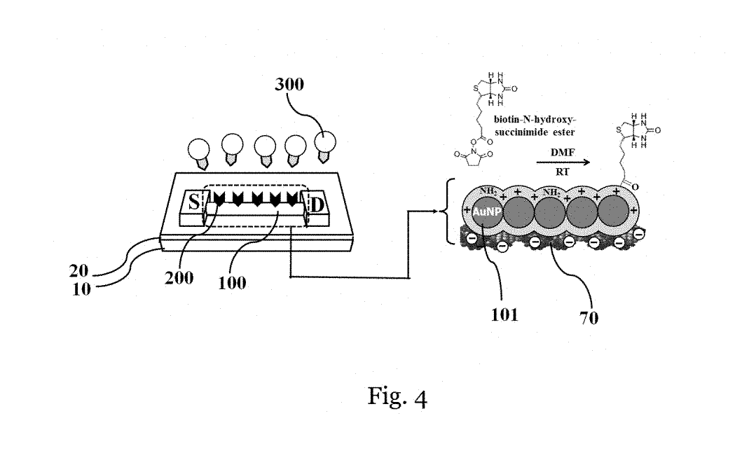

FIG. 4 is a conceptual view illustrating a state in which the protein detection receptor is fixed to the DNA-based conductive nanowire in the protein detection receptor fixing process (S300).

In the protein detection receptor fixing process (S300), a solution containing a protein detection receptor 200 may be applied to the DNA-based conductive nanowire 100 for a predetermined time to fix the protein detection receptor to the DNA-based conductive nanowire 100.

For example, when biotin-N-hydroxysuccinimide ester as the protein detection receptor 200 is fixed to the surface of the DNA-based conductive nanowire 100, the surface of the DNA-based conductive nanowire 100 may react with about 10 mM to about 20 mM of a biotin-N-hydroxysuccinimide ester solution for about 10 minutes to about 30 minutes and then be applied within about 2 minutes in each of an N,N-Dimethylformamide (DMF) buffer solution and a D.I water to fix the biotin-N-hydroxysuccinimide ester.

The protein detection receptor 200 may be a material that is capable of being specifically coupled to the target material (the detection target material), and also, it may be confirmed that the target material exists through the above-described coupling.

The protein detection receptor 200 may be a material that is generally used in the art and thus is limited to its material. For example, the protein detection receptor 200 may be proteins such as antigens, antibodies, enzymes, peptides, and polypeptides for diagnosing and preventing diseases. In more detail, the protein detection receptor 200 may be at least one of biotin, ANTI-AFP (α-fetoprotein), and ANTI-PIVKA (protein induced by vitamin K absence)-II.

The biosensor may be applicable for a receptor that detects peptide nucleic acid (PNA), locked nucleic acid (LNA), and rib nucleic acid (RNA) as well as proteins.

The ‘target material’ is not limited as long as the target material is specifically coupled to and reacts with the protein detection receptor. For example, the target material may include proteins such as antigens, antibodies, enzymes, peptides, and polypeptides.

For example, when the biotin, the ANTI-AFP (α-fetoprotein), or the ANTI-PIVKA (protein induced by vitamin K absence)-II are used as the protein detection receptors, streptavidin, AFP, and PIVKA-II, which are respectively specifically coupled to the ANTI-AFP, and the ANTI-PIVKA-II, may be used as the target materials 300.

The target material is not limited as long as the target material is specifically coupled to the detection receptor in addition to the proteins. For example, the target material 300 may include PNA, LNA, RNA, DNA, bacteria, virus, and the like.

The target material 300 may include at least one selected from avidin, neutravidin, lectin, selectin, protein A, protein G, aptamer, a tumor marker, a fluorescent molecule (Cy5, Cy3, FAM, or FITC), and a combination thereof.

The method for manufacturing the biosensor may further include an electrode formation process of connecting a source electrode coming into electrical contact with the manufactured DNA-based conductive nanowire to a drain electrode disposed to be spaced apart from the source electrode.

The electrode formation process may be performed in a pre- or post-process of the DNA alignment process (S100).

A substrate 10 used for the biosensor may include an Si wafer and a wafer on which SiO₂ is deposited, a glass substrate and a glass substrate coated with transparent conductive oxide, a flexible organic substrate such as a polymer, a metal, and the like.

An insulation layer 20 may be additionally provided to electrically isolate the substrate 10 from the electrode 30. The insulation layer 20 may be disposed on the substrate below the source electrode 31, the drain electrode 32, and the DNA-based conductive nanowire 100. When a non-conductive substrate is used as the substrate, the insulation layer may be omitted.

Various kinds of oxide films including SiO₂, Al₂O₃, Ta₂O₅, ZrO₂, HfO₂, TiO₂, and the like, various kinds of nitride films including SiON, Si₃N₄, and the like, or various kinds of Hf-based insulation films including HfSiON, HfSiOx, and the like may be used as the insulation layer 20. Also, various other materials in addition to the above-described materials may be used as the insulation layer 20.

A method for detecting a protein by using the biosensor is as follows.

When a sample including a target material comes into contact with the biosensor, the target material (the detection target material) may be specifically coupled to the protein detection receptor, and thus, an electrical signal may vary in intensity, resistance, and the like of current, which are detected through the electrode. The current may vary in intensity according to whether the target material exists and a concentration of the target material. As a result, whether the target material exists and the concentration of the target material may be detected and determined through the variation in intensity of the current.

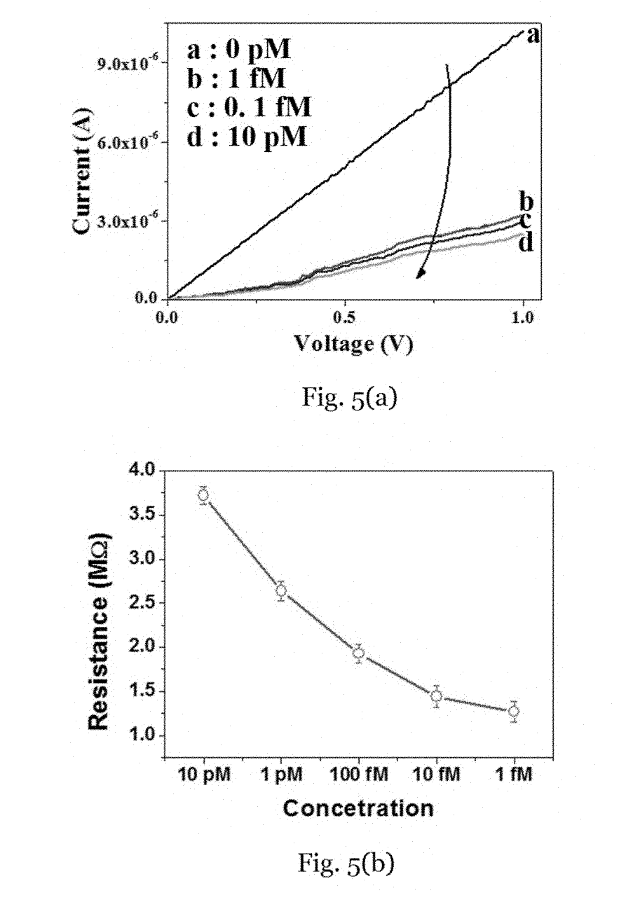

FIGS. 5(a) to 5(b) are views illustrating a variation in electrical characteristic due to whether the target material exists and the concentration of the target material according to an embodiment.

The DNA-based conductive nanowire was formed by using the gold nanoparticles to which the amine group is applied, and the biotin was prepared as the protein detection receptor. Then, streptavidin samples having concentrations (0 pM, 1 fM, 0.1 fM, and 10 pM) different from each other were applied as the target materials.

As a result, it was clearly confirmed that variations in resistance and current occurs by the coupling of the biotin and the streptavidin.

Also, it was observed that a variation in current according to the concentration of the target material.

Thus, it was confirmed that the biosensor is capable of detecting a highly sensitive and accurate biological target material, which detects a variation in electrical characteristic at a low concentration in femto (fM) and pico (pM) units.

Hereinafter, the biosensor using the DNA-based conductive nanowire is described.

The biosensor using the DNA-based conductive nanowire may include the substrate, the source and drain electrodes disposed to be spaced apart from each other on the substrate, the DNA-based conductive nanowire coming into electrical contact with the source and drain electrodes and formed by coating with the conductive nanoparticles having spontaneous positive charges, and the protein detection receptor coupled to the DNA-based conductive nanowire by the electrostatic attraction to determine the diseases at the high sensitivity.

The biosensor using the DNA-based conductive nanowire may be manufactured by the above-described method for manufacturing the biosensor using the DNA-based conductive nanowire, and thus, its detailed description will be omitted.

The DNA-based conductive nanowire may have a complex nanostructure in a nano unit, which is capable of being variously manufactured by using the electrostatic attraction and be developed for various uses such as development of a sensor, a nanowire, and an electric circuit through signal amplification.

As described above, in the biosensor using the DNA-based conductive nanowire and the method for manufacturing the same, the DNA-based conductive nanowire formed by coating with the conductive nanoparticles having spontaneous positive charges and the protein detection receptor coupled to the DNA-based conductive nanowire by the electrostatic attraction may be provided to determine the diseases at the high sensitivity.

Although the specific embodiments have been described with reference to the accompanying drawings, it will be readily understood by those skilled in the art that various modifications and changes can be made thereto without departing from the spirit and scope of the present description. Therefore, the protective range of the following claims should be construed to include many modified examples.

What is claimed is:

1. A method for manufacturing a biosensor using a DNA-based conductive nanowire, the method comprising:
a DNA alignment process of selectively aligning DNA on a substrate;
a DNA-based conductive nanowire manufacturing process in which conductive nanoparticles charged by positive charges are coupled to the aligned DNA to manufacture the DNA-based conductive nanowire having spontaneous positive charges; and
a protein detection receptor fixing process of fixing a receptor for detecting proteins to the DNA-based conductive nanowire.

2. The method of claim 1, wherein the DNA alignment process comprises:
a photoresist layer formation process of forming a photoresist layer that controls a line width of a photoresist pattern to a thickness of about 1 nm to about 10 nm after the photoresist pattern is formed on the substrate;
a nanomaterial adsorption inhibitor coating process of applying a nanomaterial adsorption inhibitor for preventing a nanomaterial from being adsorbed onto a photoresist pattern non-formation area of the substrate on which the photoresist layer is formed;
a nanomaterial adsorbent coating process in which the photoresist pattern formed on the substrate is removed, and a nanomaterial adsorbent charged by the positive charges is applied to the area of the substrate from which the photoresist pattern is removed; and
a DNA fixation process of fixing the DNA having negative charges to the substrate coated with the nanomaterial adsorbent.

3. The method of claim 2, wherein, in the photoresist layer formation process, the line width is controlled through a plasma downstream-type ashing process.

4. The method of claim 2, wherein, in the nanomaterial adsorption inhibitor coating process, the nanomaterial adsorption inhibitor comprises octadecyltrichlorosilane (OTS) or diamond like carbon (DLC).

5. The method of claim 2, wherein, in the nanomaterial adsorbent coating process, the nanomaterial adsorbent comprises aminopropyltriethoxysilane (APS) charged by the positive charges.

6. The method of claim 2, wherein, in the DNA fixation process, the substrate coated with the nanomaterial adsorbent is slanted to allow a solution containing the DNA to flow and thereby to fix the DNA.

7. The method of claim 2, wherein, in the DNA fixation process, the substrate coated with the nanomaterial adsorbent is immersed into a solution containing the DNA and then takes out to fix the DNA.

8. The method of claim 1, wherein, in the DNA-based conductive nanowire manufacturing process, the aligned DNA and the conductive nanoparticles charged by the positive charges are coupled to each other by electrostatic attraction.

9. The method of claim 1, wherein the conductive nanoparticles charged by the positive charges are spontaneously functionalized by an amine group.

10. The method of claim 1, wherein the conductive nanoparticles charged by the positive charges comprise at least one of metal particles, semiconductor particles, magnetic particles, polymer particles.

11. The method of claim 1, further comprising an electrode formation process of connecting a source electrode coming into electrical contact with the DNA-based conductive nanowire to a drain electrode disposed to be spaced apart from the source electrode.

12. The method of claim 1, wherein the protein detection receptor comprises one of biotin, anti-AFP, and anti-PIVKA-II.

13. A biosensor using the DNA-based conductive nanowire manufactured through the method claim 1.
